(19) United States
(12) Patent Application Publication
(10) Pub. No.: US 2008/0085556 A1
(43) Pub. Date: Apr. 10, 2008

(54) CULTURE DEVICE

(75) Inventors: Jason Leigh Graefing, Gleneleg (AU); Jason Paul Hayes, Heathermont (AU); David Sean O'Brien, Redland Bay (AU); Matthias Schuenemann, Brunswick (AU); Matthew Daniel Soloman, Hughesdale (AU); Jason William Spittle, Bangalow (AU)

Correspondence Address: BRINKS HOFER GILSON & LIONE/CHICAGO/COOK, PO BOX 10395, CHICAGO, IL, 60610, US

(73) Assignees: William Cook Australia Pty. Ltd., Brisbane (AU); Vance Products Incorporated, Spencer, IN (US)

(21) Appl. No.: 11/842,687

(22) Filed: Aug. 21, 2007

(30) Foreign Application Priority Data
Feb. 23, 2006 (AU) PCT/AU2006/000221
Feb. 23, 2006 (AU) 2005900835

(51) Int. Cl. C12M 1/00; C12N 5/06

(52) U.S. Cl. 435/383; 435/303.1

(57) ABSTRACT

A device for use in culturing a cell is provided. The device comprises at least one cell culture chamber and a fluid reservoir. The cell culture chamber has a tapered side wall and is in fluid connection with the fluid reservoir via a fluid path which is connected to the culture chamber via an aperture in the culture chamber. The aperture is smaller than the diameter of the cell to be cultured such that the cell is maintained within the cell culture chamber.

### CROSS-REFERENCE TO RELATED APPLICATIONS

This application is a continuation of and claims priority to PCT Application No. PCT/AU2006/000221, titled “Culture Device,” and filed on Feb. 23, 2006 and published in English on Aug. 31, 2006 as WO 2006/089354, and to Australian Application No. 2005900835, filed on Feb. 23, 2005, the entire contents of each are fully incorporated by reference herein.

### FIELD OF INVENTION

The present invention relates to a device for cell culturing. More specifically the invention relates to a microfluidic device for cell culture, more particularly for mammalian cell culture including mammalian cell replication and/or reproduction. In one application, the invention is used for culturing embryos for in vitro fertilization (IVF).

### BACKGROUND OF THE INVENTION

Microfluidics is the technology used to design, model, manufacture and mass-produce microsystems that handle fluids, gases, vapours or liquids in volumes that can be as small as nano or pico litres. Active and passive microstructures control the flow and mixing of the fluids to produce physical, chemical, biochemical and microbiological reactions in a rapid, cost-effective manner. Microfluidics have a range of applications, including the culture of cells and the automation of highly manual laboratory processes, such as in vitro fertilization (IVF) onto a single substrate.

Culturing of distinct cells, or distinct populations of cells such as embryos, in a single culture device provides certain difficulties. It has been demonstrated that the exchange of certain autocrine, paracrine and endocrine molecules improves the success rate of culturing cells, and a fluidic communication between distinct cells is considered beneficial to the cells in a culture environment. However, benefits also exist in the separation of cells and the provision of a unique cell distinct from other cells in the culture environment.

Physical barriers that allow fluidic communication between cells, but restrict intermingling of cells have been considered. A physical barrier provides the benefits of the exchange of autocrine, paracrine and endocrine molecules, and the maintenance of distinct cells. However, the provision of physical barriers has required the use of large fluid volumes, and the exchange of medium between the physical barriers often results in the cells being dislodged from their desired location. Physical barriers also often result in dead volumes and trapped gas bubbles that hamper fluid transport. Furthermore, physical barriers may also increase the time required to exchange culture medium during the culturing process, and where no fluidic communication exists between distinct cells, the exchange of medium can be laborious as well as exposing the cells to mechanical, thermal and chemical stress.

The present invention seeks to overcome, or at least minimise, the problems associated with the prior art.

### SUMMARY OF THE INVENTION

In a first aspect, the present invention provides a device for use in culturing a cell, the device comprising at least one cell culture chamber and a fluid reservoir, the cell culture chamber having a tapered side wall and being in fluid connection with the fluid reservoir via a fluid path, the fluid path being connected to the culture chamber via an aperture in the culture chamber, the aperture being smaller than the diameter of the cell to be cultured such that the cell is maintained within the cell culture chamber.

In a further aspect, the present invention provides a method of culturing a cell, the method comprising providing culture medium in the at least one cell culture chamber of a device according to the first aspect of the invention and incubating the device.

### BRIEF DESCRIPTION OF THE DRAWINGS

FIG. 1 is a schematic perspective view of a device in accordance with the invention.

FIG. 1B shows a schematic plan view of the device from the top.

FIG. 1C shows a schematic plan view of the device from the bottom.

FIG. 1D shows a schematic cross-section through AA′ of the device of FIG. 1B.

FIG. 2 is a cross-sectional view through a cell culture chamber.

FIG. 3A shows a side view of a cross-section of the device.

FIG. 3B shows a side view of a cross section of the device.

FIG. 4A shows a schematic plan view of the cell culture area.

FIG. 4B shows a schematic plan view of the cell culture camber.

FIG. 4C, 4D and 4E show a schematic cross-sectional view of variations of the device.

FIGS. 5A, 5B, 5C, 5D, 5E, 5F, 5G and 5H show a side cross-sectional view of a device in accordance with the invention.

FIG. 6A-6J show a side cross sectional view of a device in accordance with the invention.

FIG. 7 shows examples of labelling of cell culture chambers.

FIG. 8A shows a schematic perspective view of the base plate.

FIG. 8B shows a schematic plan view of the base plate.

FIG. 8C shows a schematic cross-section through M′ of the device of FIG. 8B.

FIG. 9A shows a schematic perspective view of the cell culture device assembly.

FIG. 9B shows a schematic cross-section of the cell culture device assembly.

### DETAILED DESCRIPTION OF THE PREFERRED EMBODIMENTS

The device of the present invention provides co-culturing of a distinct cell, such as a cell that may give rise to an embryo, so that a common culture medium can be used. The present device also provides a physical barrier to inhibit co-mingling of embryos. The present device also provides for the changing of culture medium to be effected gradually so as to reduce the shock from a sudden change in culture conditions.

In a first aspect, the present invention provides a device for use in culturing a cell, the device comprising at least one cell culture chamber and a fluid reservoir, the cell culture chamber having a tapered side wall and being in fluid connection with the fluid reservoir via a fluid path, the fluid path being connected to the culture chamber via an aperture in the culture chamber, the aperture being smaller than the diameter of the cell to be cultured such that the cell is maintained within the cell culture chamber.

Without being limiting, the provision of a cell culture chamber having a tapered side wall allows for easier access by operators and/or allows for better quality optical imaging.

In a preferred embodiment, the tapered side wall of the cell culture chamber is tapered at an angle of about 450 to about 600 relative to the horizontal axis of the cell culture chamber.

In another preferred embodiment, the cell culture chamber has a further side wall that is tapered at an angle of about 800 to about 900 relative to a horizontal axis of the cell culture chamber.

In a further preferred embodiment, the device comprises multiply fluidically connected cell culture chambers.

In yet a further embodiment, the device comprises multiple fluid reservoirs, wherein each fluid reservoir is in fluid connection with each other as well as the cell culture chambers.

In another preferred embodiment, the fluid reservoir has a loading port with a wall that mates with a device, such as a liquid introduction device. More preferably, the fluid reservoir has a loading port with a tapered wall that mates with a device, such as a liquid introduction device

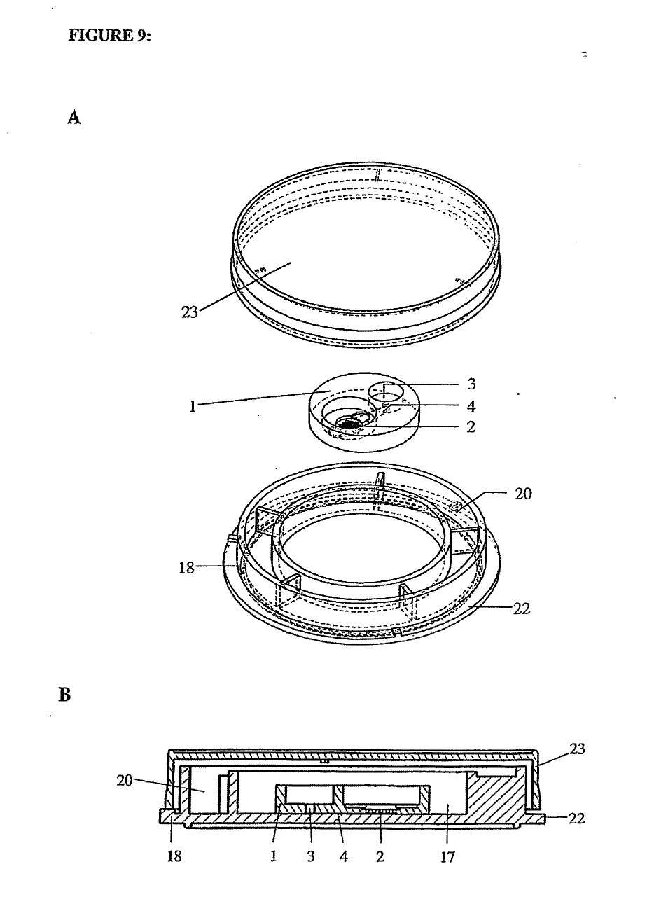

In a preferred embodiment, the device 1 may be assembled into a base plate 18 and covered with a lid 23 (see FIG. 9A).

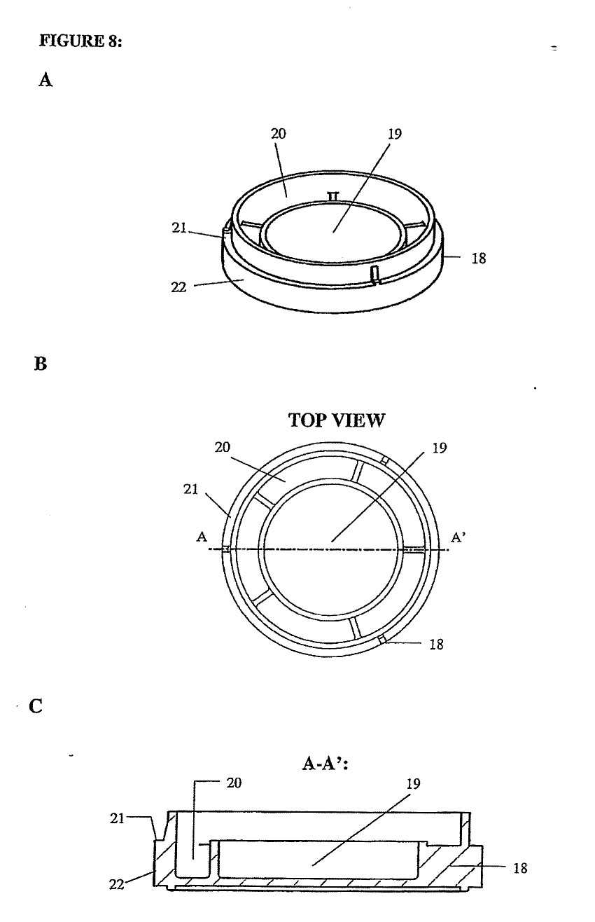

The base plate 18 comprises an assembly area 19 and a series of one to seven fluid reservoirs 20 (see FIG. 8A, 8B).

The fluid reservoirs 20 may be used to pre-condition temperature and pH value of culture or process medium required for subsequent culture steps during incubation.

The diameter of the assembly area 19 may be larger than the diameter of the device 1, thus creating a spill channel 17 at the periphery of the device 1 to collect medium overflowing from the cell culture chamber and/or the fluid reservoir (see FIG. 9B).

The base plate 18 comprises an outer rim 22 with a vertical dimension large enough to enable a secure and stable grip by an operator. More preferably, the vertical dimension of the outer rim 22 is 3 to 8 mm (see FIG. 8C).

The diameter of the lid 23 may be the same size or smaller than the diameter of the outer rim 22 of the base plate 18 (see FIG. 9B) in order to improve the handling of the assembled device by the operator and to reduce the risk of dropping the assembled device during manipulation.

In assembled state, the lid 23 rests on the lid ridge 21 of the base plate 18, leaving the outer rim 22 accessible for gripping and handling.

In a further preferred embodiment, the cell culture chamber is labelled with a label based on a symbol code. Preferably, the symbol code is readable for both right and left-handed operation of the device. More preferably, the symbol code is readable from above or below the device.

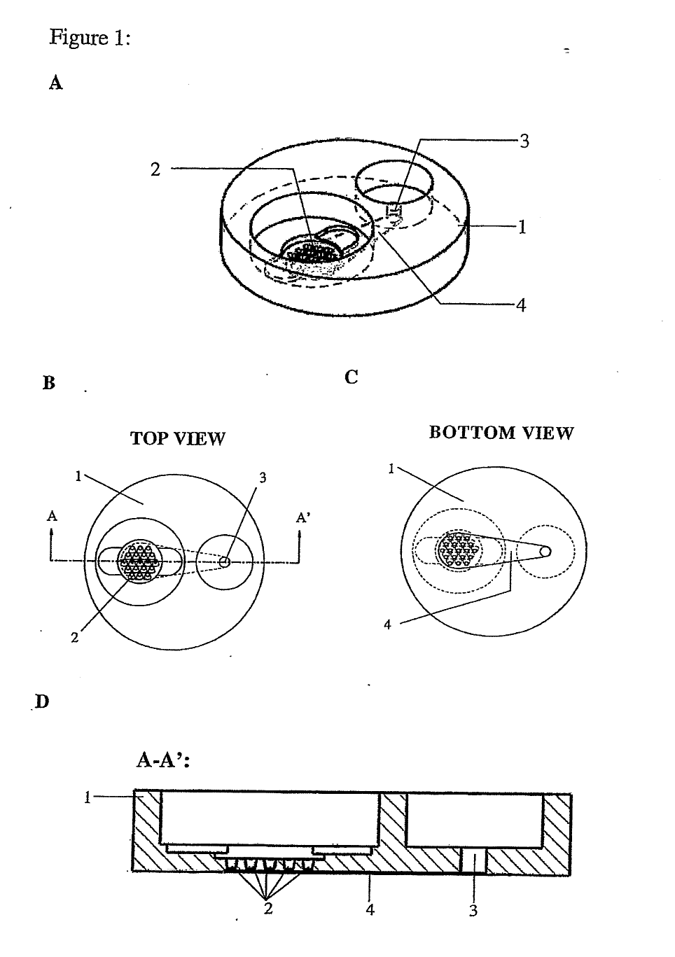

Referring now to the drawings, FIG. 1A is a perspective view of a device according to the present invention. The device 1 includes a plurality of cell culture chambers 2 and a fluid reservoir 3. FIG. 1D is a cross section through AA′ of the device 1 of FIG. 1B and shows a fluid path 4 fluidically connecting the reservoir 3 with cell culture chambers 2.

A variety of materials can be used to manufacture the device of the invention. Preferably, the device 1 is manufactured from materials selected from the group comprising cyclic olefin copolymer (COC), polycarbonate (PC), polystyrene (PS), polymethyl-methacrylate (PMMA), polyethyleneterephthalate (PET), polyimide (PI), polyetherimide (PEI), polydimethylsiloxane (PDMS), acrylonitrile butadiene styrene (ABS), cellulose acetate (CA), cellulose acetate butyrate (CAB), high density polyethylene (HDPE), low density polyethylene (LDPE), polyamide (PA), polybutylene terephtalate (PBT), polyether ether ketone (PEEK), polyethylene terephtalate glycol (PETG), polymethylpentene (PMP), polyoxide methylene (POM), polypropylene (PP), polysulfone (PSU), polytetrafluoroethylene (PTFE), polyvinylchloride (PVC), polyvinylidene chloride (PVDC) or polyvinylidene fluoride (PVDF) or combinations thereof. Preferably the material selected has low water vapour permeability, low water absorption and for optical transparency. The carbon dioxide permeability of the material will typically depend on the process requirements.

One or all parts of the device may be coated on one or all surfaces with a barrier layer, such as Parylene, in order to render the bulk material non-cytotoxic, to lower the water absorption of the bulk material, to lower the water vapour absorption of the bulk material, and to protect the bulk material from potential harmful interaction with culture medium or culture oil.

The device according to the present invention may be manufactured using microfabrication techniques known to those of skill in the art including molding techniques such as hot embossing, stamping or injection molding, or by polymerizing the precursor polymers within the mold. The device 1 may be fabricated as a single item or be comprised of multiple parts, for example a structured part comprising the fluid paths and structures, a spacer and a part comprising a base plate, that combine to form the device 1 of the invention.

The structured part may be made by micromilling or similar mechanical microfabrication. In a preferred embodiment, the cell culture chamber 2 and the fluid reservoir 3 being milled on one face and the fluid path 4 on the other face.

Where the device 1 is comprised of separate parts, the parts may be connected using an adhesive, or by other bonding methods such as thermal diffusion bonding, ultrasonic bonding, solvent bonding and laser welding.

In a preferred embodiment, the fluid path 4 may be achieved using a prestructured spacer with a thickness equivalent to the desired thickness of the fluid path 4. For example, the fluid path 4 may be achieved by the use of a thermally activated tape or a pressure-activated tape (e.g. Scotch tape), which defines the fluid path 4 as well as providing the mechanical bond between the structured part and the base part.

Spacer structures may also be integrated into either the base part or the structured part or into both the base part and the structured part.

The device of the present invention may be used for culturing a single cell per cell culture chamber 2, or a population of cells in each cell culture chamber 2. In a preferred embodiment, a single cell may be cultured in the cell culture chamber.

The cell culture chamber 2 functions to restrict movement of a cell and retain the cell in the cell culture chamber 2. The cell culture chamber also maintains a unique address of cells, facilitates cell communication via autocrine, paracrine and endocrine molecule exchange, and facilitates medium exchange.

In a preferred embodiment, the hydrophilicity of the microfluidic surfaces may be improved by surface treatment techniques such as plasma polymerisation, UV treatment, saponification, polyethylene oxide grafting, surface texturing or electrowefting may be applied.

Preferably, the volume of the cell culture chamber 2 is about 0.1 μ1 to 1.5 μ1. More preferably, the volume of the cell culture chamber 2 is 0.1 to 0.3 μ1. Most preferably, the volume of the cell culture chamber 2 is 0.125 μl.

In a preferred embodiment, the device 1 comprises multiple fluidically connected cell culture chambers 2. For ease of loading and unloading of cells, it is preferred that multiple cell cultures chambers 2 are orientated in the same direction (see FIGS. 1A, 1B, 1C & 4A).

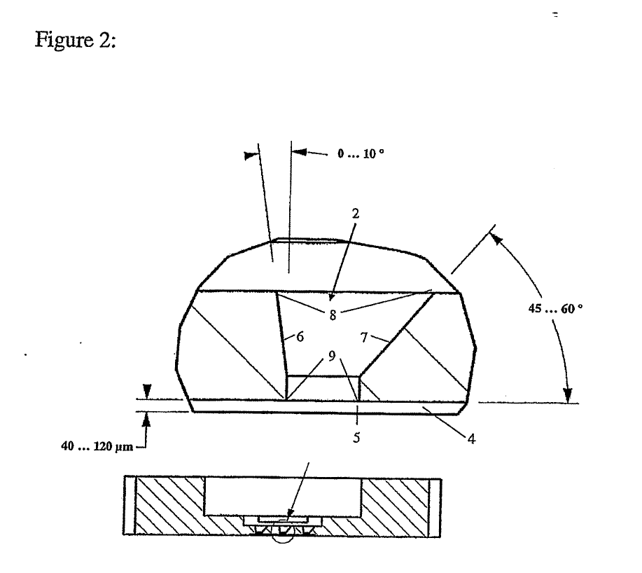

FIG. 2 is a cross section through a cell culture chamber 2 showing the preferred size of the base of the cell culture chamber 2. The aperture 5 connecting the cell culture chamber 2 with the fluid path 4, may be of any shape. Preferably, the diameter of the fluid path 4 is 40 to 120 μm. The cell culture chamber 2 has a tapered side wall 6 which is preferably tapered at an angle of about 45° to about 60° relative to the horizontal axis of the cell culture chamber 2. The cell culture chamber 2 typically also has another side wall 7 that is slightly tapered at angle of about 80° to about 90° relative to the horizontal axis of the cell culture chamber 2. The tapering of the side walls allows for ease of manufacture, inhibits gas bubble formation and/or assists in optical inspection. The upper 8 and lower 9 rims of the cell culture chamber 2 are typically rounded off to, for example, assist fluid flow and/or the avoidance of gas bubbles.

The aperture 5 in the cell culture chambers 2 (FIG. 2) can be achieved by providing overlapping milling depth (e.g. the sum of the milling depth for the ceil culture chambers 2 and the milling depth for the fluid path 4 is larger than the thickness of the structured part) or by drilling the aperture 5 between the two milled structures. The drilling may be achieved by, for example, conventional mechanical drilling or by laser microstructuring. Alternatively, the aperture 5 during formation of the cell culture chambers by injection molding (e.g. by placing an appropriately structured insert into the injection mold to form the aperture 5).

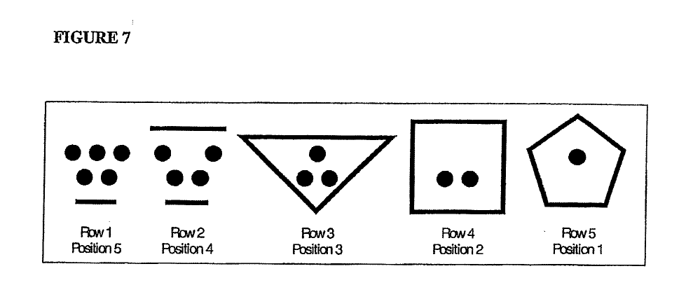

In a preferred embodiment, the cell culture chamber 2 is labelled. Labelling of the cell culture chamber 2 may be by an alphanumeric code, symbol-based code, bar code, dot code, or by any other such means that are readable, preferably by optical inspection by either inverted or conventional microscopes (see FIG. 7). Preferably, the labels are readable for both left- and right-handed operation of the device, and/or for reading from above or below.

In a preferred embodiment, the cell culture chamber 2 has a diameter at its base of about 200 μm to about 600 μm. More preferably, the cell culture chamber 2 has a diameter at its base of about 300 μm to about 500 μm.

Preferably, multiple fluid reservoirs are provided in the device 1, wherein each fluid reservoir is preferably in fluid connection with each other as well as the cell culture chambers 2. The fluid reservoir 3 can be used for both the ingress and egress of fluid from the culture chambers 2.

The fluid reservoir 3 may comprise a luer locking fitting at its upper rim, an o-ring around its upper ring, a tapered entry port mating with preferred liquid introduction device (e.g. laboratory pipette) and/or a temporary sealing device around its upper ring, to assist with exchanging fluids in the fluid reservoir 3.

The fluid reservoir 3 may have a wicking device, such as a sponge, integrated. Preferably, the wicking device is integrated so that the position of the underside of the wicking device defines the desired filling height of the cell culture chambers 2.

In a preferred embodiment, a fluid volume indicator such as steps or markings is formed on the wall of the fluid reservoir 3 to indicate fluid volumes.

In a preferred embodiment, the cell culture medium is injected into the fluid reservoir 3. From the fluid reservoir 3, the cell culture medium flows, preferably by capillary flow or by applied pressure difference, via the fluid path 4, to the aperture 5 of the cell culture chambers 2, and subsequently fills the cell culture chambers 2. The fluid level in the cells culture chambers 2 will typically depend directly on the injected fluid volume. The fluid levels may be equilibrated by, for example, gravity.

Without being limited by theory, filling the cell culture chambers 2 with culture medium from above would almost certainly trap gas bubbles in the cell culture chambers 2, and therefore inhibit successful cell culturing. Filling cell culture chambers 2 from below overcomes the problem of trapped gas bubbles.

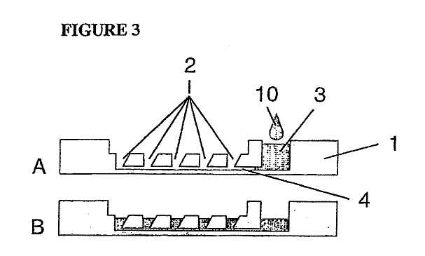

FIGS. 3A and 3B shows a side view of a cross section of the device 1 and in FIG. 3A shows medium 10 applied to fluid reservoir 3. FIG. 3B shows medium 10 flowing from fluid reservoir 3 through fluid path 4, and filling cell culture chambers 2 from below.

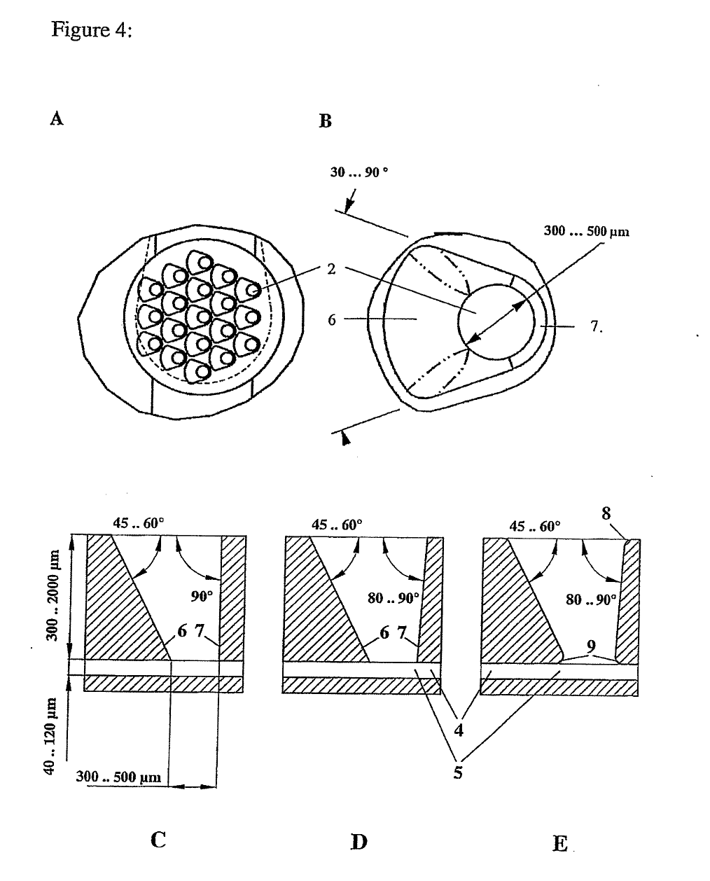

FIG. 4B is a schematic plan view of a cell culture chamber 2 and shows that the cell culture chamber 2 typically has at least two side walls inclined at an angle of about 30° to about 90°.

FIGS. 4C, 4D and 4E is a schematic cross section through a cell culture chamber 2 and shows different configuration for the tapered side wall 6 which typically is tapered at an angle of about 45° to about 60° relative to the horizontal axis of the cell culture chamber 2. The cell culture chamber 2 typically also has another side wall 7 that is slightly tapered at angle of about 80° to about 90° relative to the horizontal axis of the cell culture chamber 2. FIG. 4E shows a variation wherein the upper 8 and lower 9 rims of the cell culture chamber 2 are typically rounded off. The typical depth, 300 to 2000 μm, of the cell culture chamber 2 is also indicated.

In a preferred embodiment, in use, the medium in the cell culture chamber 2 is substantially covered by a substance, for example, a cell culture oil such as a paraffin based oil, such as Cook® Sydney IVF Culture Oil or a silicone-based oil, to minimize evaporation. It is preferred that if culture oil is used, that cells do not come into contact with the culture oil.

Preferably, the medium in the fluid reservoir 3 is also substantially covered by a substance, for example, a cell culture oil such as a paraffin-based oil, such as Coolc® Sydney IVF Culture Oil or a silicone-based oil, to minimize evaporation.

In a further aspect, the present invention provides a method of culturing a cell, the method comprising providing culture medium in the at least one cell culture chamber of a device according to the first aspect of the invention and incubating the device.

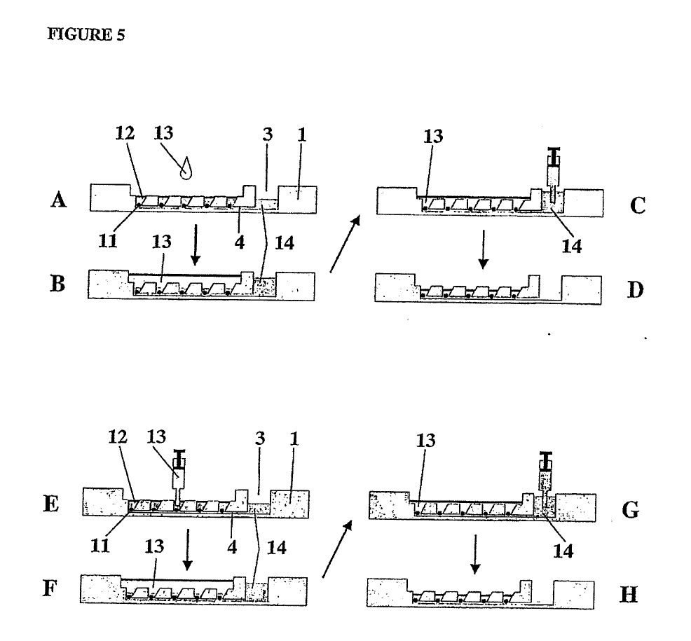

FIG. 5 is a side cross sectional view of the device 1 and schematic flow chart for a preferred method of changing medium in the cell culture chambers 2. FIG. 5 shows a population of cells 11, such as an embryo, located in the cell culture chambers 2. In FIG. 5A, cell culture oil 12 has been layered onto the cell culture medium 14 in the cell culture chamber 2 so that the culture oil 12 substantially covers the surface of the culture medium. Substitute medium 13 is applied to the cell culture chambers 2 and diffuses through the culture oil 12 (FIG. 5B). The original medium 14 is drawn out of the cell culture chamber 2 via the aperture in the cell culture chamber 2, and moves through the fluid path 4 to the fluid reservoir 3. The original medium 14 is drawn out of the cell culture chamber 2 by removal of the original medium 14 from the fluid reservoir 3 by, for example, pipetting or use of a syringe (FIG. 5C). Once a substantial proportion of the original medium 14 has been removed, the level of medium in the cell culture chamber 2 returns to the desired level.

In a preferred embodiment, substitute medium 13 is injected below the below the culture oil layer by, for example, a syringe or pipette (FIG. 5E, 5F). The original medium 14 is drawn out of the cell culture chamber 2 via the aperture in the cell culture chamber 2, and moves through the fluid path 4 to the fluid reservoir 3. The original medium 14 is drawn out of the cell culture chamber 2 by removal of the original medium 14 from the fluid reservoir 3 by, for example, pipetting or use of a syringe (FIG. 5G). Once a substantial proportion of the original medium 14 has been removed, the level of medium in the cell culture chamber 2 returns to the desired level (FIG. 5H).

In a preferred embodiment, the cell culture medium is introduced into, and removed from, the fluid reservoir 3, by pipetting, wicking or pumping.

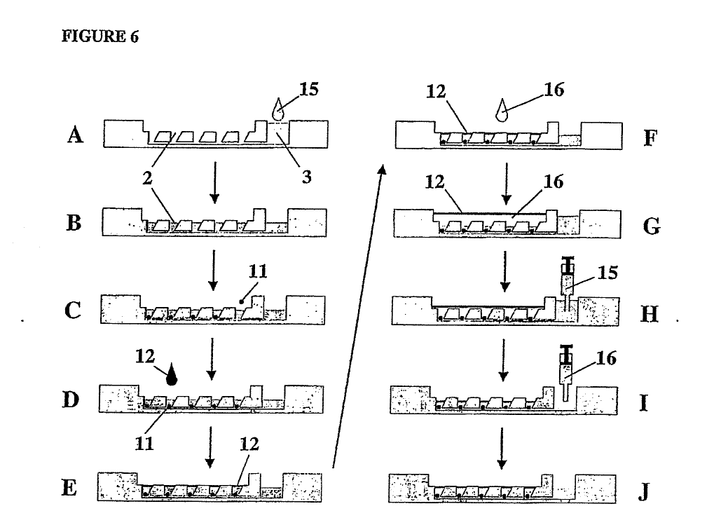

The following is a general description of one manner of using the device of the invention for in vitro growth of embryos prior to implantation of the embryo into a recipient's reproductive tract with reference to FIG. 6.

A defined volume of Cook® Sydney IVP cleavage medium 15 (Cooke Sydney IVF Cleavage Medium is a complex bicarbonate buffered medium that contains antioxidants, non-essential amino acids, and human serum albumin, but no glucose (see table 1)) is injected into the fluid reservoir 3. From there, it fills the cell culture chambers 2 from below, either by capillary flow, or by applied pressure. The medium volume is chosen so that the upper level of fluid is below the upper rim of the cell culture chambers 2.

The embryos are then pipetted into the cell culture chambers 2. An IVF culture oil such as a paraffin- or silicone-based oil 12 is applied to the cell culture chamber 2 area and fluid reservoir 3 so that it substantially covers the surface of the medium in the cell culture chamber 2 and fluid reservoir 3. The device 1 is then incubated for the required time (typically 48 to 72 hrs).

A defined volume of Cook® Sydney IVF blastocyst medium 16 (Cook® Sydney IVF Blastocyst Medium is a bicarbonate buffered medium that contains essential and nonessential amino acids, glucose and human serum albumin to promote the development of early embryos to blastocysts (see table 2)) is applied to the cell culture chamber 2.

Due to the lower specific weight, the IVF culture oil 12 will rise to the top of the Cook® Sydney IVF Blastocyst medium 16 layer. An amount of medium is then extracted from the fluid reservoir 3, preferably by wicking, pipetting or pumping.

The process may be repeated until all Cook® Sydney IVF cleavage medium is either completely removed or diluted to the required volume, for example 5% of the total volume of cell culture medium.

After the culture medium exchange, the oil layer 12 settles back on top of the medium in the cell culture chambers 2. The device 1 is then incubated for the required time (typically 48 to 72 hrs). The embryos may extracted as required from the cell culture chamber 2 by, for example, pipetting.

In another embodiment, a defined volume of Cook® Sydney IVP cleavage medium 15 is injected into the fluid reservoir 3. From the fluid reservoir 3, the medium fills the cell culture chambers 2 from below, either by capillary flow, or by applied pressure. The medium volume is chosen so that the upper level of fluid is below the upper rim of the cell culture chambers 2. An IVF culture oil, such as paraffin- or silicone-based oil 12 is applied to the cell culture chamber 2 and the fluid reservoir 3. The device is then thermally and chemically equilibrated (typically in an incubator). The embryos are then pipetted into the cell culture chambers 2 below the oil layer. The device 1 is then suitably incubated for the required time (typically 48 to 72 hrs).

In another embodiment, a defined volume of Cook® Sydney IVF cleavage medium 15 is injected into the fluid reservoir 3. From the fluid reservoir 3, the medium fills the cell, culture chambers 2 from below, either by capillary flow, or by applied pressure. No additional IVF culture oil is being used. The device is then thermally and chemically equilibrated (typically in an incubator). The embryos are then pipetted into the cell culture chambers 2. The device 1 is then suitably incubated for the required time (typically 48 to 72 hrs).

TABLE 1
Cook ® Sydney IVF Cleavage Medium

| | | |
|---|---|---|
| Sodium Chloride | Gentamicin | L-Asparagine |
| | | Monohydrate |
| Potassium Chloride | EDTA Disodium Salt | L-Aspartic Acid |
| Potassium Dihydrogen | L-Taurine | L-Glutamic acid |
| Orthophosphate | | |
| Magnesium Sulphate | L-Glutamine | L-Proline |
| Sodium Hydrogen | Glycine | L-Serine |
| Carbonate | | |
| Sodium Pyruvate | Processed Water | Human Serum |
| | | Albumin |
| Hemi-Calcium Lactate | L-Alanine | |

TABLE 2
Cook ® Sydney IVF Blastocyst Medium

| | | |
|---|---|---|
| Sodium Chloride | L-Taurine | L-Methionine |
| Potassium Chloride | Processed | L-Tyrosine |
| | Water | Disodium |
| | | Dihydrate |
| Potassium Dihydrogen | Glycine | L-Tryptophan |
| Orthophosphate | | |
| Magnesium Sulphate | L-cysteine | L-Alanine |
| | Disodium | |
| | Monohydrate | |
| Sodium Hydrogen | L-Arginine | L-Valine |
| Carbonate | | |
| Sodium Pyruvate | L-Isoleucine | L-Aspartic Acid |
| Hemi-Calcium Lactate | L-Histidine | L-Asparagine |
| | | Monohydrate |
| Gentamicin | L-Lysine | L-Glutamic acid |
| D-Glucose | L-Leucine | L-Serine |
| L-Glutamine | L-Phenylalanine | L-Proline |
| | Human Serum | |
| | Albumin | |

The suitability of the invention was verified by attempting to grow mouse embryos from 2-cell stage (an embryo that contains only 2 equally sized cells) to blastocyst stage (an embryo at approximately 4 days of age with a blastocoelic cavity surrounded by a thin layer of cells) following the protocols described above.

A device according to the description was manufactured from polymethyl-methacrylate (PMMA) by micromilling. Parts were coated with Parylene C and bonded together by thermal diffusion bonding. The device was cleaned by rinsing with purified water to remove any potential particulate contaminants. Female mice are superovulated and mated with stud males. They are sacrificed the following day and the 2-cell embryos removed from the fallopian tubes. These embryos were placed into the cell culture chambers of the device, which had been filled with equilibrated culture medium. At the same time, around 20 2-cell embryos are placed into a standard cell culture dish, which had been filled with a reasonable amount of the same equilibrated culture medium. The embryos in the standard cell culture dish serve as control sample to account for variations in embryo viability and environmental conditions.

Both test and control sample (the device according to the invention and the standard cell culture dish, respectively) were then incubated in the same incubator for 72 hours at 37° C. in a 5-6% CO₂ enriched atmosphere. After 72 hours, the developmental stage of each embryo was recorded and the percentage of embryos having reached the blastocyst stage was calculated separately for test sample and control sample. The percentage results for the test sample are then normalised with the percentage results for the control sample.

If the normalised percentage of 2-cell embryos having reached the blastocyst stage of development is 80% or higher, the device is deemed to have passed the mouse embryo assay. The prototype of the device according to the invention has fulfilled this criterion and therefore has passed the test.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A device for use in culturing a cell, the device comprising at least one cell culture chamber and a fluid reservoir, the cell culture chamber having a tapered side wall and being in fluid connection with the fluid reservoir via a fluid path, the fluid path being connected to the culture chamber via an aperture in the culture chamber, the aperture being smaller than the diameter of the cell to be cultured such that the cell is maintained within the cell culture chamber.

2. The device according to claim 1, wherein the device comprises multiple fluidically connected cell culture chambers.

3. The device according to claim 2, wherein the device comprises multiple fluid reservoirs, wherein each fluid reservoir is in fluid connection with each other as well as the cell culture chambers.

4. The device according to claim 1, wherein the device is manufactured from materials selected from the group comprising cyclic olefin copolymer (COC), polycarbonate (PC), polystyrene (PS), polymethyl-methacrylate (PM MA), polyethyleneterephthalate (PET), polyimide (PI), polyetherimide (PEI), polydimethylsiloxane (PDMS), acrylonitrile butadiene styrene (ABS), cellulose acetate (CA), cellulose acetate butyrate (CAB), high density polyethylene (HDPE), low density polyethylene (LDPE), polyamide (PA), polybutylene terephthalate (PBT), polyether ether ketone (PEEK), polyethylene terephtalate glycol (PETG), polymethylpentene (PMP), polyoxide methylene (POM), polypropylene (PP), polysulfone (PSU), polytetrafluoroethylene (PTFE), polyvinylchloride (PVC), polyvinylidene chloride (PVDC) or polyvinylidene fluoride (PVDF) or combinations thereof.

5. The device according to claim 4, wherein one or all parts of the device is coated on one or all surfaces with a barrier layer such as parylene.

6. The device according to claim 1, wherein the tapered side wall is tapered at an angle of about 45° to about 60° relative to a horizontal axis of the cell culture chamber.

7. The device according to claim 1, wherein the cell culture chamber has a further side wall that is tapered at angle of about 80° to about 90° relative to a horizontal axis of the cell culture chamber.

8. The device according to claim 1, wherein the fluid reservoir has a loading port with a tapered wall that mates with a liquid introduction device.

9. The device according to claim 1, wherein the cell culture chamber is labeled with a label based on a symbol code, wherein the symbol code is readable for both right and left-handed operation of the device.

10. The device according to claim 9, wherein the symbol code is readable from above or below the device.

11. A method of culturing a cell comprising the steps of:
providing at least one cell culture chamber and a fluid reservoir, with the cell culture chamber comprising a tapered side wall and a fluid path providing fluid communication between the cell culture chamber and the fluid reservoir;
providing an aperture defined in the cell culture chamber in communication with the fluid path, wherein the aperture includes a smaller diameter than a diameter of a cell to be cultured to maintain the cell within the chamber;
providing a cell culture medium within the cell culture chamber; and
incubating the cell culture chamber.

12. The method of claim 11, wherein the cell culture chamber is filled from below.
